SEQUENCE LISTING (4 sequences)

SEQ ID NO: 1 - protein, 9 aa, Artificial Sequence
  KRAS G12D 9mer
GADGVGKSA

SEQ ID NO: 2 - protein, 10 aa, Artificial Sequence
  KRAS G12D 10mer
GADGVGKSAL

SEQ ID NO: 3 - protein, 366 aa, Homo sapiens [sequence omitted]

SEQ ID NO: 4 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

(19) United States
(12) Patent Application Publication
(10) Pub. No.: US 2019/0175709 A1
(43) Pub. Date: Jun. 13, 2019

(54) GENETIC EXPRESSION OF HLA MOLECULES TO ENHANCE IMMUNOTHERAPIES

(71) Applicant: Providence Health & Services - Oregon, Portland, OR (US)

(72) Inventor: Eric Tran, Portland, OR (US)

(73) Assignee: Providence Health & Services - Oregon, Portland, OR (US)

(21) Appl. No.: 16/207,648

(22) Filed: Dec. 3, 2018

Related U.S. Application Data
(60) Provisional application No. 62/596,321, filed on Dec. 8, 2017.

(51) Int. Cl. A61K 39/00; A61P 35/00; A61P 35/04

(52) U.S. Cl. CPC A61K 39/001111; A61K 39/001154; A61K 2039/53; A61P 35/04; A61K 2039/5158; A61P 35/00

(57) ABSTRACT

Methods of treating a subject with a tumor, for example in combination with cancer immunotherapy are provided. In some embodiments, the methods include obtaining one or more samples including tumor cells from the subject and measuring human leukocyte antigen (HLA) and/or β2-microglobulin (B2M) expression level, genotype, and/or copy number in the tumor cells. One or more HLA and/or B2M alleles with reduced expression, function, and/or copy number in the tumor are selected and a nucleic acid encoding the one or more HLA and/or B2M alleles is administered to the subject. One or more cancer immunotherapies are also administered to the subject.

### CROSS REFERENCE TO RELATED APPLICATION

This claims the benefit of U.S. Provisional Application No. 62/596,321, filed Dec. 8, 2017, which is incorporated herein by reference in its entirety.

### FIELD

This disclosure relates to methods of cancer immunotherapy, particularly utilizing HLA expression to enhance immunotherapy.

### BACKGROUND

Immunotherapy approaches have generated high expectations for increasing success rates in cancer treatment. However, immune evasion (e.g., avoidance of T cell recognition) is common in malignant cells. Thus, methods to improve cancer immunotherapy, including overcoming immune evasion are needed.

### SUMMARY

One mechanism of immune evasion in tumor cells is down-regulation, decreased function, or loss of expression of human leukocyte antigen (HLA). Therefore, increasing or restoring HLA expression or function is described herein as a strategy for improving the efficacy of immunotherapy approaches to treating cancer. By determining the specific HLA allele(s) with reduced expression or function in the subject's tumor, immune response to the tumor (such as an immunotherapy) can be restored.

Disclosed herein are methods of treating a subject with a tumor, for example in combination with cancer immunotherapy. In some embodiments, the methods include obtaining one or more samples of a tumor from the subject and measuring human leukocyte antigen (HLA) and/or β2-microglobulin (B2M) expression level, genotype, and/or copy number in the tumor. One or more HLA and/or B2M alleles having reduced expression, function (e.g., due to mutation), and/or copy number in the tumor are selected and a nucleic acid encoding the one or more selected HLA and/or B2M alleles is administered to the subject. In some embodiments, one or more cancer immunotherapies are also administered to the subject. In one example, the cancer immunotherapy is adoptive T cell therapy, for example administering to the subject T cells that recognize a tumor antigen in the subject's tumor in the context of the administered HLA molecule.

The foregoing and other features of the disclosure will become more apparent from the following detailed description, which proceeds with reference to the accompanying figures.

### BRIEF DESCRIPTION OF THE DRAWINGS

FIG. 1 is a schematic diagram showing an exemplary protocol for carrying out the disclosed methods.

FIGS. 2A and 2B are graphs showing T-cell recognition of tumor cell lines after transfection of HLA-C*08:02 constructs. T cells were genetically engineered to express a T-cell receptor (TCR) that specifically recognizes a 9 amino acid (9mer) or 10 amino acid (10mer) mutated KRAS-G12D epitope when presented in the context of the HLA-C*08:02 molecule. The KRAS-G12D 9mer (FIG. 2A) and 10mer (FIG. 2B) reactive T cells were then cocultured with pancreatic cancer cells lines (MDA-Panc48, HPAC, and Panc-1), which endogenously express the KRAS G12D mutation but not HLA-C*08:02, that had been transfected with nothing (Mock), or RNA that encoding the HLA-C*08:02 molecule or HLA-C*08:02 in combination with β2-microglobulin (HLA-C*08:02+B2M). After an overnight coculture, cells were collected and analyzed for T-cell activation (4-1BB expression). Data was gated on CD8⁺ T cells expressing the KRAS-G12D reactive TCRs.

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

The Sequence Listing is submitted as an ASCII text file in the form of the file named Sequence_Listing.txt, which was created on Dec. 3, 2018, and is ˜4.8 kilobytes, which is incorporated by reference herein.

SEQ ID NOs: 1 and 2 are KRAS G12D peptides.

SEQ ID NO: 3 is the amino acid sequence of a HLA-C*08:02 protein.

SEQ ID NO: 4 is the amino acid sequence of an exemplary B2M protein.

### DETAILED DESCRIPTION

Most T cells recognize short peptide antigens that are presented by human leukocyte antigens (HLA) expressed by target cells. Tumors often have downregulated or genetic loss of HLA class-I molecules which render them difficult to target by cytotoxic T cells. In addition to these defects, the tumor microenvironment contains barriers and many factors that are hostile and suppressive to anti-tumor T cells. Disclosed herein are methods to enhance the effectiveness of immunotherapies, especially against cancer, by overcoming the above challenges.

Advantages of the methods disclosed herein include harnessing two elements of T cell recognition of antigens, HLA and T cells, which are important to T cell-based killing of tumor cells, and it overcomes multiple mechanisms used by tumor cells to evade the immune response, such as downregulation or loss of HLA, the active suppression of anti-tumor T cells through immune checkpoint molecules, and low frequency of tumor-reactive T cells often found in patients with cancer. In addition, the disclosed methods do not utilize molecules with toxicity to normal tissue. Furthermore, the methods to deliver HLA and optionally B2M do not require high tumor cell specificity (in contrast with toxic compounds), as the HLA utilized is normal (wild type) for the treated subject and the tumor killing is mediated through T cells that are specific for tumor antigens (in the case of adoptive T cell immunotherapies).

### I. Terms

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. For example, the term “a probe” includes single or plural probes and can be considered equivalent to the phrase “at least one probe.” As used herein, the term “comprises” means “includes.” Thus, “comprising a probe” means “including a probe” without excluding other elements. It is further to be understood that all base sizes or amino acid sizes, and all molecular weight or molecular mass values, given for nucleic acids or polypeptides are approximate, and are provided for descriptive purposes, unless otherwise indicated.

All publications, patent applications, patents, and other references mentioned herein are incorporated by reference in their entirety. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

To facilitate review of the various embodiments of the disclosure, the following explanations of terms are provided:

Expression Control Sequences: Nucleic acid sequences that regulate the expression of a heterologous nucleic acid sequence to which it is operably linked. Expression control sequences are operably linked to a nucleic acid sequence when the expression control sequences control and regulate the transcription and, as appropriate, translation of the nucleic acid sequence. Thus, expression control sequences can include appropriate promoters, enhancers, transcription terminators, a start codon (e.g., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components, for example, leader sequences and fusion partner sequences. In one example, expression control sequences include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included.

Heterologous: Originating from a different genetic sources or species. For example, a nucleic acid that is heterologous to a cell originates from an organism or species other than the cell in which it is expressed. Methods for introducing a heterologous nucleic acid into host cells includes, for example, transformation with a nucleic acid, including electroporation, lipofection, and/or particle gun acceleration.

In another example of use of the term heterologous, a nucleic acid operably linked to a heterologous promoter is from an organism or species other than that of the promoter, or is a promoter that is not normally linked to the nucleic acid in the organism or species. In other examples of the use of the term heterologous, a nucleic acid encoding a polypeptide or portion thereof is operably linked to a heterologous nucleic acid encoding a second polypeptide or portion thereof, for example to form a non-naturally occurring fusion protein.

Human Leukocyte Antigen (HLA): A gene complex on human chromosome 6, encoding major histocompatibility complex (MHC) proteins. As used herein, the term “HLA” typically refers to MHC class I molecules (HLA-A, HLA-B, and HLA-C). It may also be used to refer to MHC class II molecules, or the HLA gene complex; this can be determined by the context.

MHC class I molecules present epitopes typically derived from endogenous proteins for presentation to cytotoxic T lymphocytes (CTLs). HLA-A, HLA-B and HLA-C molecules bind peptides of about eight to ten amino acids in length that have particular anchoring residues. The anchoring residues recognized by an HLA Class I molecule depend upon the particular allelic form of the HLA molecule. A CD8⁺ T cell bears T cell receptors that recognize a specific epitope when presented by a particular HLA molecule on a cell. When a CTL precursor that has been stimulated by an antigen presenting cell to become a cytotoxic T lymphocyte contacts a cell that bears such an HLA-peptide complex, the CTL forms a conjugate with the cell and destroys it. MHC class I HLAs associate with β2-microglobulin (B2M), which is encoded by a gene on human chromosome 15.

MHC class II molecules (HLA-DR, HLA-DQ, and HLA-DP in humans) present epitopes typically derived from exogenous proteins for presentation to T helper cells. The complex of an MHC class II protein and its ligand, typically a peptide of 9-21 amino acids in length, constitutes a ligand for the T-cell receptor (TCR). These antigens can stimulate CD4⁺ T cells to activate other cells of the immune system.

Immunotherapy: A therapy that increases immune system response to a disease, such as cancer. Types of immunotherapy include monoclonal antibodies, immune checkpoint inhibitors (for example, PD-1 inhibitors, PD-L1 inhibitors, or CTLA-4 blockade), dendritic cell therapies, adoptive T cell therapy, and tumor vaccines (such as tumor cell vaccines or antigen vaccines).

Inhibiting or treating a disease: Inhibiting a disease, such as tumor growth, in some examples refers to inhibiting the full development of a disease. In several examples, inhibiting a disease refers to lessening symptoms of a tumor in a person who is known to have cancer, or lessening a sign or symptom of the tumor. “Treatment” refers to a therapeutic intervention that ameliorates a sign or symptom of a disease or pathological condition related to the disease, such as a tumor.

Isolated: An “isolated” biological component (such as an HLA nucleic acid, a tumor antigen polypeptide, a T cell, or other biological component) has been substantially separated or purified away from other biological components in which the component naturally occurs, such as other chromosomal and extrachromosomal DNA, RNA, and proteins. Nucleic acid molecules, polypeptides, and cells that have been “isolated” include nucleic acid molecules purified by standard purification methods. The term also embraces nucleic acid molecules and polypeptides prepared by recombinant expression in a host cell as well as chemically synthesized molecules. Isolated does not require absolute purity, and can include biological components that are at least 50% isolated, such as at least 75%, 80%, 90%, 95%, 98%, 99% or even 100% isolated.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Subject: A living multi-cellular vertebrate organism, a category that includes human and non-human mammals.

T cell: A white blood cell involved in the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, are involved in antibody responses as well as killer T cell responses. In one embodiment, a CD4⁺ T cell is a CD4⁺ regulatory T cell. CD8⁺ T cells carry the “cluster of differentiation 8” (CD8) marker. In one embodiment, a CD8⁺ T cell is a cytotoxic T lymphocyte (CTL). In another embodiment, a CD8⁺ T cell is a suppressor T cell.

Vector: A nucleic acid molecule allowing insertion of foreign or heterologous nucleic acid into a cell without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and/or translation of an inserted gene or genes. In some non-limiting examples, the vector is a viral vector, such as a retroviral vector, adenoviral vector, or lentiviral vector.

### II. Methods of Improving Immunotherapy

Disclosed herein are methods for treating a subject with cancer, e.g. by enhancing or improving an immunotherapy treatment. In some embodiments, the methods enhance cancer immunotherapy, particularly in a subject with a decrease or loss of expression and/or function of one or more HLA molecules or a decrease in B2M in a tumor.

The methods include identifying changes in expression and/or function of HLA or B2M molecules in a sample of a tumor from a subject. Following selection of an HLA or B2M molecule with altered expression (such as decreased expression or loss of expression) or function (such as one or more mutations) in the tumor, nucleic acid encoding the HLA, and optionally β2-microglobulin (B2M)), is introduced to the tumor cells in vivo, followed by, or in conjunction with, treatment of the subject with an immunotherapy such as adoptive T-cell therapy or immune checkpoint blockade.

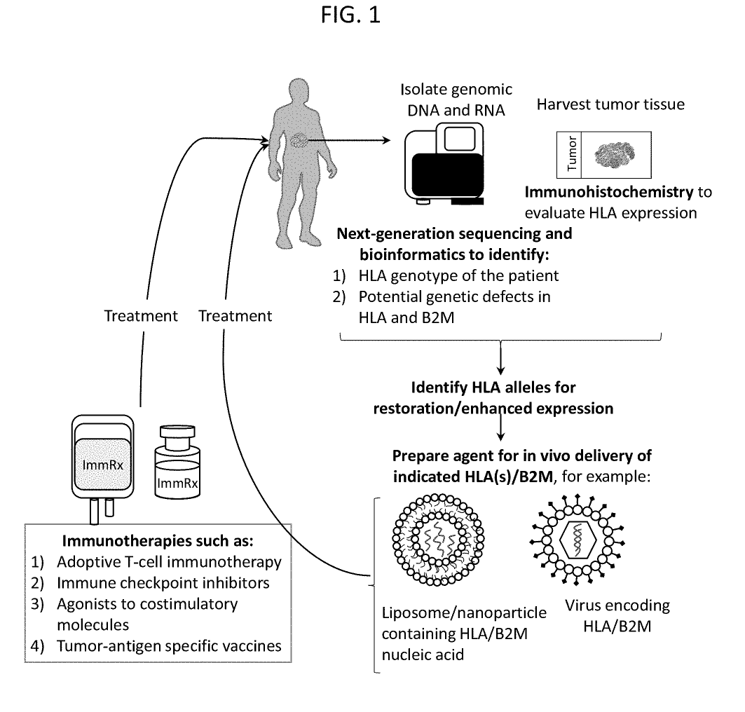

In some embodiments, the methods include obtaining one or more samples including tumor or cancer cells (such as solid tumor cells or hematological malignancy cells) from the subject and measuring human leukocyte antigen (HLA) and/or β2-microglobulin (B2M) expression level, genotype, and/or copy number in the tumor or cancer cells. One or more HLA and/or B2M alleles with reduced expression and/or copy number or with one or more mutations in the tumor or cancer cells are selected and a nucleic acid encoding the one or more selected HLA and/or B2M alleles is administered to the subject. One or more cancer immunotherapies are also administered to the subject. FIG. 1 provides a non-limiting example of the disclosed methods.

In some examples, the subject has cancer, such as a solid tumor or a metastasis of a solid tumor. Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma, and retinoblastoma). In non-limiting examples, solid tumors that can be treated or inhibited by the methods disclosed herein include colorectal cancer, pancreatic cancer, non-small cell lung cancer (NSCLC), or metastases thereof. In particular examples, the subject has a tumor that expresses KRAS G12D or KRAS G12V.

In other examples, the subject has a hematological malignancy. Examples of hematological malignancies include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), T-cell large granular lymphocyte leukemia, polycythemia vera, lymphoma, diffuse large B-cell lymphoma, Hodgkin's lymphoma, non-Hodgkin's lymphoma (indolent and high grade forms), mantle cell lymphoma, follicular cell lymphoma, multiple myeloma, Waldenstrom's macroglobulinemia, heavy chain disease, myelodysplastic syndrome, hairy cell leukemia and myelodysplasia. In non-limiting examples, hematological malignancies that can be treated or inhibited by the methods disclosed herein include acute myeloid leukemia (AML), acute lymphoblastic leukemia (ALL), chronic lymphocytic leukemia (CLL), chronic myeloid leukemia (CML), non-Hodgkin lymphoma (NHL), and myelodyplastic syndrome.

### A. Identification of HLA Expression in Tumor Cells

The methods disclosed herein include identifying changes in HLA expression and/or function in a tumor in a subject (such as downregulated expression, altered function, somatic mutations, and/or loss of heterozygosity). In some examples, the HLA molecule is a class I MHC molecule (e.g., HLA-A, HLA-B, HLA-C, and/or B2M). In other examples, the HLA molecule is a class II MHC molecule. As discussed below, following identifying one or more changes in HLA expression and/or function in the tumor, the subject is treated to increase or restore HLA expression or function in the tumor.

The HLA genes are found in a block on human chromosome 6 (6p21). The HLA locus includes six protein coding MHC class I genes (HLA-A, HLA-B, HLA-C, HLA-E, HLA-F, and HLA-G) and 12 protein coding MHC class II genes. Over 3000 HLA allele sequences have been identified (Shiina et al., J. Hum. Genet. 54:15-39, 2009). The B2M gene is on human chromosome 15 (15q21.1).

In some embodiments, a tumor sample is obtained from the subject with the tumor and HLA expression in the tumor and/or any genetic changes (e.g., mutation or loss of heterozygosity) in HLA or B2M genes in the tumor is evaluated. The tumor sample may be a primary tumor sample (including a solid tumor sample or a hematological malignancy sample) or a metastatic tumor sample. In some examples the tumor sample is from a progressing tumor or a regressing tumor (e.g., a sample from a tumor following at least one treatment). For example, a sample from a tumor can be obtained by surgical excision of all or part of the tumor or by collecting a fine needle aspirate from the tumor.

In some examples, nucleic acids (such as DNA and/or RNA) are isolated from the tumor sample for analysis of HLA and/or B2M. In other examples, tumor samples are prepared by fixing and embedding tissue in a medium and tissue sections are prepared on a solid support, or include a cell suspension prepared as a monolayer on a solid support (such as a glass slide), for example by smearing or centrifuging cells onto the solid support. In additional examples, fresh frozen (for example, unfixed) tissue or tissue sections may be used in the methods disclosed herein.

In some embodiments, HLA and/or B2M protein levels are measured, for example, using one or more immunoassays. Exemplary immunoassays include immunohistochemistry and flow cytometry. In other embodiments, HLA and/or B2M RNA levels are measured, for example, using microarray hybridization, in situ hybridization, or Northern blotting. In still further examples, HLA allele type and/or presence of mutations in HLA alleles and/or B2M are determined by sequencing (for example, Sanger sequencing or next generation sequencing techniques). In additional examples, HLA and/or B2M allele copy number is determined. Exemplary methods for determining allele copy number include fluorescent in situ hybridization (FISH) comparative genomic hybridization (CGH), and whole exome sequencing. PCR-based methods can also be used to detect HLA allele type, expression level, or loss of heterozygosity (LOH).

Examples of software that can be used to determine HLA alleles of the subject from next generation sequencing include PHLAT (Bai et al., BMC Genomics 15:325, 2014), HLAreporter (Huang et al., Genome Med. 7:25, 2015), HLAscan (Ka et al., BMC Bioinformatics 18:258, 2017), OptiType (Szolek et al., Bioinformatics 30:3310-3316, 2014), HLA-MA (Messerschmidt et al., Bioinformatics 33:2241-2242, 2017), HLAminer (Warren et al., Genome Med. 4:95, 2012), and ATHLATES (Liu et al., Nucleic Acids Res. 41:e142, 2013). Examples of software that can be used to determine allele copy numbers from next generation sequencing include Sequenza (Favero et al., Ann. Oncol. 26:64-70, 2015) and LOHHLA (loss of heterozygosity in human leukocyte antigen, which specifically evaluates LOH at the HLA locus) (McGranahan et al., Cell 171:1259-1271, 2017).

The methods include selecting one or more HLA and/or B2M alleles with reduced expression and/or copy number in the tumor sample. An allele with reduced expression in the tumor sample includes an allele with at least 10% decreased expression (for example, at least 15%, 20%, 25%, 30%, 40%, 50%, 60%, 70%, 80%, 90%, 95%, or even 100% decreased expression) in at least some cells in the tumor sample compared to expression of the allele in a control. In some examples, the control is expression of the same HLA in normal (e.g., non-tumor) cells from the subject. In other examples, the control can be a reference value, such as an average expression level for normal (non-tumor) cells in a population. An allele with reduced copy number in the tumor sample includes an allele with a reduced number of copies compared to a control, for example, having only a single copy (e.g., loss of heterozygosity) or no copies in at least some cells in the tumor sample. In some examples, a tumor is identified as having a reduced copy number of an HLA allele if it is present in less than 90% of the tumor cells examined (such as less than 80%, 70%, 60%, 50%, 40%, 30%, 20%, or 10% of tumor cells, for example, 10-90%, 25-75%, 40-80%, or 20-50% of tumor cells).

In some embodiments, the methods further include identifying one or more immunogenic neoepitopes (such as an epitope with a tumor-specific mutation) in the tumor sample from the subject. Neoepitopes are identified by identifying mutations occurring in the tumor, for example, by whole exome sequencing and/or transcriptome sequencing of nucleic acids isolated form the tumor sample. Nucleic acids encoding the mutations (for example, the mutation flanked by 12 amino acids on either side) are expressed in autologous antigen presenting dendritic cells (DCs). Tumor infiltrating lymphocyte (TIL) cultures isolated from the tumor are cocultured with the DCs and T cell reactivity is determined (for example using ELISPOT or flow cytometric analysis). Alternatively, DCs are pulsed with peptides including the identified mutations and then cocultured with TILs to identify reactive T cells. In other examples, T cells from sources other than the tumor, such as peripheral blood, are used to identify reactive T cells.

In other examples, other tumor-specific antigens (e.g., non-mutated antigens) are targeted. For example, oncoviral antigens such as human papilloma virus (HPV) which is associated with cervical and some head and neck cancers, and cancer germline (CG) antigens such as NY-ESO-1, MAGEC1, MAGEA3, SSX-2, and KK-LC-1 (CT83) which are expressed by a subset of a variety of cancers, are targeted.

In additional embodiments, the methods further include identifying the HLA genotype of the subject with the tumor. The HLA genotype of the subject is determined by sequencing (such as next generation sequencing). In some examples, the HLA genotype of the subject is compared to the HLA alleles expressed in the tumor sample, for example, to determine down-regulation or loss of expression of an HLA allele in the tumor.

In one non-limiting example, the HLA allele having reduced expression, function, and/or copy number is HLA-C*08:02. An exemplary amino acid sequence of HLA-C*08:02 includes or consists of:

(SEQ ID NO: 3)
MRVMAPRTLILLLSGALALTETWACSHSMRYFYTAVSRPGRGEPR
FIAVGYVDDTQFVQFDSDAASPRGEPRAPWVEQEGPEYWDRETQK
YKRQAQTDRVSLRNLRGYYNQSEAGSHTLQRMYGCDLGPDGRLLR
GYNQFAYDGKDYIALNEDLRSWTAADKAAQITQRKWEAAREAEQR
RAYLEGTCVEWLRRYLENGKKTLQRAEHPKTHVTHHPVSDHEATL
RCWALGFYPAEITLTWQRDGEDQTQDTELVETRPAGDGTFQKWAA
VVVPSGEEQRYTCHVQHEGLPEPLTLRWGPSSQPTIPIVGIVAGL
AVLAVLAVLGAVMAVVMCRRKSSGGKGGSCSQAASSNSAQGSDES
LIACKA

In other non-limiting examples, the HLA allele having reduced expression, function, and/or copy number is HLA-A*11:01. In additional examples, the HLA allele having reduced expression, function, and/or copy number is HLA-A*02:01 (e.g., for restricted epitopes derived from the shared antigens HPV-16 E6 and E7 proteins, NY-ESO-1, MAGE-C2, and SSX-2), HLA-B*15 (e.g., for restricted epitopes from shared cancer germline antigen KK-LC-1 (CT83)), HLA-A*01 (e.g., for restricted epitopes from MAGE-A1, MAGE-A3, and KK-LC-1 (CT83)), HLA-B*35:01, and B*40:01 (e.g., for restricted epitopes from HPV-16 E6 and E7 proteins).

An exemplary B2M amino acid sequence includes or consists of:

(SEQ ID NO: 4)
MSRSVALAVLALLSLSGLEAIQRTPKIQVYSRHPAENGKSNFLNC
YVSGFHPSDIEVDLLKNGERIEKVEHSDLSFSKDWSFYLLYYTEF
TPTEKDEYACRVNHVTLSQPKIVKWDRDM

### B. Expression of HLA Molecules in Tumor Cells

The methods include increasing expression of the HLA and/or B2M alleles selected in Section IIA as having reduced expression, function, and/or copy number in the tumor. The HLA allele(s) selected will vary, depending on the subject and the tumor. A nucleic acid encoding the selected HLA allele(s) is prepared, and optionally a nucleic acid encoding B2M is prepared, if B2M is also to be administered to the subject. The nucleic acid(s) can be cloned into a suitable plasmid or viral vector. In some examples, mRNA of the HLA alleles(s), and optionally B2M is synthesized (e.g., using chemical synthesis or in vitro transcription of a DNA encoding the alleles(s)). In other examples, the selected HLA allele(s) are cloned from the subject. In one non-limiting example, the selected HLA allele encodes HLA-C*08:02 (e.g., SEQ ID NO: 3) and/or the B2M nucleic acid encodes the amino acid sequence of SEQ ID NO: 4.

In some embodiments, a selected HLA nucleic acid is administered to the subject to deliver the nucleic acid to the tumor. In other embodiments, both a selected HLA nucleic acid and a B2M nucleic acid are administered to the subject. The nucleic acid(s) can be administered to the subject systemically or locally. In some examples, the nucleic acid(s) are administered to the subject systemically, for example, parenterally (such as intravenously, intracranially, or intraperitoneally). In other examples, the nucleic acid(s) are administered to the subject locally, for example by injection into the tumor (e.g., intratumorally) or to a site in close proximity to the tumor. In some examples, the route of administration is selected based on the type or location of the tumor and/or metastases.

In some embodiments, the nucleic acids are delivered in a composition including a pharmaceutically acceptable carrier or excipient. Pharmaceutically acceptable carriers include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in the composition. Other excipients include, but are not limited to, detergents, proteins, e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400, PEG-600, PEG-1000, PEG-1450, PEG-3350, PEG-6000, PEG-8000 and any molecular weights in between these values, propylene glycols (PG), sugar alcohols, such as a carbohydrate, for example sorbitol. The detergent, when present, can be an anionic, a cationic, a zwitterionic or a nonionic detergent. In some embodiments, the detergent is a nonionic detergent. In some examples, the nonionic detergent is a sorbitan ester, for example, polyoxyethylenesorbitan monolaurate (TWEEN-20) polyoxyethylenesorbitan monopalmitate (TWEEN-40), polyoxyethylenesorbitan monostearate (TWEEN-60), polyoxyethylenesorbitan tristearate (TWEEN-65), polyoxyethylenesorbitan monooleate (TWEEN-80), polyoxyethylenesorbitan trioleate (TWEEN-85). In specific examples, the detergent is TWEEN-20 and/or TWEEN-80.

In some embodiments, the HLA nucleic acid(s) are delivered to tumor cells using a viral vector. In some examples, the HLA nucleic acid(s) are inserted into a viral vector and are operably linked to a promoter that directs expression of the nucleic acid(s) in a cell, such as a tumor cell. In some non-limiting examples, the promoter is a constitutive promoter (e.g., cytomegalovirus (CMV), SV40, phosphoglycerate kinase (PGK), ubiquitin C (UBC), elongation factor-1 (EFS), chicken β-action short promoter (CBH), EF-1 alpha (EF1a) promoter, or EF1a short promoter (EFS)), or an inducible or tissue-specific promoter. The vector may also include additional expression control elements, such as one or more enhancers, leader sequences, transcription terminators, start and/or stop codons, and polyadenylation signals.

In further embodiments, the HLA nucleic acid(s) are delivered to tumor cells using an oncolytic virus. In some examples, the HLA nucleic acid(s) (and optionally B2M nucleic acid) is inserted in an oncolytic virus for administration to the subject.

In other embodiments, the HLA nucleic acid(s) are delivered to tumor cells using a nanoparticle or liposome delivery system. In some examples, an RNA encoding the HLA nucleic acid(s) (for example, in vitro transcribed RNA) is incorporated into a nanoparticle or liposome for administration to the subject.

Multiple doses of the HLA nucleic acid(s) can be administered to the subject. For example, the HLA nucleic acid(s) can be administered daily, every other day, twice per week, weekly, every other week, every three weeks, monthly, or less frequently. A skilled clinician can select an administration schedule based on the subject, the condition being treated, the previous treatment history, and other factors.

1. Viral Vectors

Viral vectors suitable for delivery of HLA nucleic acid(s) to tumor cells include retrovirus, adenovirus, adeno-associated virus (AAV), vaccinia virus, fowlpox, and lentivirus vectors. In particular non-limiting examples disclosed herein, the nucleic acids are delivered to tumor cells with a viral vector including one or more HLA-encoding nucleic acids. In some embodiments, a viral vector including one or more B2M-encoding nucleic acids is also delivered to the tumor cells. The HLA and B2M nucleic acids may be included in the same vector or in separate vectors. In examples where an HLA nucleic acid and a B2M nucleic acid are included in the same vector, the nucleic acids can be separated by a sequence that allows for cleavage into the HLA and B2M proteins after translation (such as furin-SGSG-P2A sequence). In other examples where an HLA nucleic acid and a B2M nucleic acid are included in the same vector, the nucleic acids can be separated by an internal ribosome entry sequence (IRES) to allow expression of both nucleic acids.

a. Lentivirus Vectors

In some examples, a lentivirus vector is used. Lentiviruses are a genus of retroviruses characterized by a long incubation period and the ability to infect non-dividing cells. Lentiviruses are complex retroviruses, which, in addition to the common retroviral genes gag, pol, and env, contain other genes with regulatory or structural function. Examples of lentiviruses include HIV, SIV, FIV, SIV, BIV, CAEV and EIAV.

Lentiviral vectors have been generated by deleting the genes env, vif, vpr, vpu and nef to make lentiviral vectors safe as gene therapy vectors for human use. Lentiviral vectors integrate stably into chromosomes of target cells, which is required for long-term expression, and they do not transfer viral genes, therefore avoiding the problem of generating transduced cells that can be destroyed by cytotoxic T lymphocytes. In addition, lentiviral vectors have a relatively large cloning capacity. For example, vectors derived from HIV-1 allow efficient in vivo and ex vivo delivery, integration and stable expression of transgenes into cells such a neurons, hepatocytes, and myocytes (Blomer et al., J Virol 71:6641-6649, 1997; Kafri et al., Nat Genet 17:314-317, 1997; Naldini et al., Science 272:263-267, 1996; Naldini et al., Curr Opin Biotechnol 9:457-463, 1998).

The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTRs promote transcription and polyadenylation of the virion RNAs. The LTR contains all other cis-acting sequences necessary for viral replication. Lentiviruses also have additional genes, including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors, packaging cell lines and methods of generating lentiviral gene therapy vectors are described in Escors and Breckpot, Arch Immunol Ther Exp 58(2):107-119, 2010; Naldini et al., Science 272:263-267, 1996; Naldini et al., Proc Natl Acad Sci USA 93:11382-11388, 1996; Naldini et al., Curr Opin Biotechnol 9:457-463, 1998; Zufferey et al., Nat Biotechnol, 15:871-875, 1997; Dull et al., J Virol 72: 8463-8471, 1998; Ramezani et al., Mol Ther 2:458-469, 2000; and U.S. Pat. Nos. 5,994,136; 6,013,516; 6,165,782; 6,207,455; 6,218,181; 6,218,186; 6,277,633; 7,901,671; 8,551,773; 8,709,799; and 8,748,169, which are herein incorporated by reference in their entirety.

b. AAV Vectors

In other embodiments, an adenoviral vector or adeno-associated virus (AAV) vector is used. In particular embodiments, the vector is an AAV vector. AAV is a small, non-enveloped helper-dependent parvovirus classified in genus Dependoparvovirus of family Parvoviridae. AAV has a linear, single-stranded DNA genome of about 4.7 kb. The genome is flanked by inverted terminal repeats (ITRs) flanking two open reading frames (ORFs), rep and cap. The rep ORF encodes four replication proteins (Rep78, Rep68, Rep52, and Rep4) and the cap ORF encodes three viral capsid proteins (VP1, VP2, and VP3) and an assembly activating protein (AAP). AAV requires a helper virus (such as adenovirus, herpes simplex virus, or other viruses) to complete its life cycle. AAV is currently in use in numerous gene therapy clinical trials worldwide. Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. AAV possesses several desirable features for a gene therapy vector, including the ability to bind and enter target cells, enter the nucleus, the ability to be expressed in the nucleus for a prolonged period of time, and low toxicity. Because of the advantageous features of AAV, in some embodiments the present disclosure uses AAV for delivery of HLA nucleic acid molecules in methods disclosed herein.

The ITRs are the only component required for successful packaging of a heterologous protein in an AAV capsid. In some examples, the AAV vector includes 5′ and 3′ ITRs flanking an HLA nucleic acid (and optionally a B2M nucleic acid) operably linked to a promoter. The vector may also include additional elements, such as an enhancer element (e.g., a nucleic acid sequence that increases the rate of transcription by increasing the activity of a promoter) and/or a polyadenylation signal. In particular examples, the enhancer is a cytomegalovirus (CMV) enhancer or a woodchuck post-transcriptional regulatory element (WPRE). Exemplary promoters include a chicken (3-actin (CBA) promoter, a ubiquitous promoter (such as a glucuronidase beta (GUSB) promoter), or a neuronal-specific promoter (such as platelet-derived growth factor B chain (PDGF-beta) promoter or neuron-specific enolase (NSE) promoter). In additional examples, the polyadenylation signal is a 3-globin polyadenylation signal, an SV40 polyadenylation signal, or a bovine growth hormone polyadenylation signal. Other elements that optionally can be included in the vector include tags (such as 6×His, HA, or other tags for protein detection). Any combination of ITRs, enhancers, promoters, polyadenylation signals, and/or other elements can be used.

The AAV serotype can be any suitable serotype for delivery of transgenes to a subject. In some examples, the AAV vector is a serotype 9 AAV (AAV9). In other examples, the AAV vector is a serotype 5 AAV (AAV5). In other examples the AAV vector is a serotype 1, 2, 3, 4, 6, 7, 8, 10, 11 or 12 vector (i.e. AAV1, AAV2, AAV3, AAV4, AAV6, AAV7, AAV8, AAV10, AAV11 or AAV12). In yet other examples, the AAV vector is a hybrid of two or more AAV serotypes (such as, but not limited to AAV2/1, AAV2/7, AAV2/8 or AAV2/9). The selection of AAV serotype will depend in part on the cell type(s) that are targeted for gene therapy.

c. Oncolytic Viruses

In further embodiments, the HLA nucleic acid(s) are delivered to tumor cells using an oncolytic virus. In some examples, the HLA nucleic acid(s) (and optionally B2M nucleic acid) is inserted in an oncolytic virus for administration to the subject. Exemplary oncolytic viruses include adenoviruses, vaccinia virus, measles virus, coxsackievirus, poliovirus, reovirus, parvovirus, vesicular stomatitis virus, Newcastle disease virus, Maraba virus, and Echovirus. Oncolytic viruses provide some tumor-cell selectivity and most oncolytic viruses in clinical development are non-integrating.

2. Microparticle Delivery

In some embodiments, the HLA (and optionally B2M) nucleic acids are administered using biodegradable microparticles (˜1-100 μm) or nanoparticles (˜50-1000 nm). Nanoparticles and microparticles (also known as nanospheres or microspheres) are drug delivery vehicles that can carry encapsulated molecules such as synthetic small molecules, proteins, peptides, cells, and/or nucleic acids for either rapid or controlled release. A variety of molecules (e.g., proteins, peptides and nucleic acid molecules) can be efficiently encapsulated in nano/microparticles.

The nano/microparticles for use with the methods described herein can be any type of biocompatible particle, for example biodegradable particles, such as polymeric particles, including polyamide, polycarbonate, polyalkene, polyvinyl ethers, and cellulose ether nano/microparticles. In some embodiments, the particles are made of biocompatible and biodegradable materials. In some embodiments, the particles include, but are not limited to particles comprising poly(lactic acid) or poly(glycolic acid), or both poly(lactic acid) and poly(glycolic acid). In particular embodiments, the particles are poly(D,L-lactic-co-glycolic acid) (PLGA) particles. Additional nano/microparticles include biodegradable poly(alkylcyanoacrylate) particles (Vauthier et al., Adv. Drug Del. Rev. 55: 519-48, 2003).

Various types of biodegradable and biocompatible nano/microparticles, methods of making such particles, including PLGA particles, and methods of encapsulating a variety of synthetic compounds, proteins and nucleic acids are described in U.S. Patent Publication No. 2007/0148074; U.S. Publication No. 20070092575; U.S. Patent Publication No. 2006/0246139; U.S. Pat. Nos. 5,753,234; 7,081,489; and PCT Publication No. WO/2006/052285).

3. Liposomal Delivery

In some embodiments, the HLA (and optionally B2M) nucleic acids are administered using liposomes. The nucleic acid(s) are incorporated into a liposome for delivery to the subject. Liposomes include a lipid bilayer surrounding a cavity in which a molecule, such as a nucleic acid, can be encapsulated. The liposome can be unilamellar (having a single lipid bilayer membrane) or oligolamellar or multilamellar (having multiple, usually concentric, membrane layers and are typically larger than 0.1 μm). Liposomes are formulated to carry agents, such as nucleic acids, either contained within the aqueous interior space (water soluble active agents) or partitioned into the lipid bilayer (water-insoluble active agents). Exemplary liposome-forming lipids include phospholipids, glycolipids and sphingolipids, for example, phosphatidylcholine, phosphatidylserine, phosphatidylinositol, phosphatidylglycerol, diphosphatidylglycerol and N-acyl phosphatidylethanolamine. In other examples, liposomes are composed of 3β-[N—(N′,N′-dimethylaminoethane carbamoyl] cholesterol (DC-Chol), 1,2-dioleoyl-sn-glycero-3-phosphoethanolamine (DOPE), or N-[1-(2,3-dioleoloxy)propyl]-N,N,N-trimethyl ammonium chloride (DOTAP). Combinations of lipids can also be used in liposomes. In additional examples, modified liposome-forming lipids, such as PEGylated lipids are used to form liposomes for nucleic acid delivery.

Lipid nanoparticles or nanoemulsions may also be used to administer the HLA (and optionally B2M) nucleic acid(s) (see, e.g., Xue et al., Curr. Pharm. Des. 21:3140-3147, 2015).

### C. Immunotherapies

One or more immunotherapies is administered to the subject with the tumor in addition to administration of the selected HLA nucleic acid(s). The immunotherapy may be administered to the subject prior to, simultaneously or substantially simultaneously with, or after the selected HLA nucleic acid(s) are administered.

In some embodiments, the selected HLA nucleic acid(s) are administered to the subject prior to the immunotherapy. In some examples, the HLA nucleic acid(s) can be administered to the subject about 1 to 72 hours prior to the immunotherapy (e.g., 1-12 hours before, 4-18 hours before, 6-24 hours before, 18-48 hours before, or 24-72 hours before). In other examples, the HLA nucleic acid(s) are administered to the subject about 1 to 21 days prior to the immunotherapy (e.g., 1-4 days before, 3-10 days before, 5-14 days before, 8-16 days before, or 12-21 days before). The amount of time between administration of the HLA nucleic acid(s) and the immunotherapy can be selected based in part on the expected amount of time needed for HLA expression in the tumor. In some embodiments, the HLA nucleic acid(s) are also administered periodically during administration of immunotherapy, and may also be administered until tumor clearance or progression.

Cancer immunotherapies of use in the disclosed methods include monoclonal antibodies, immune checkpoint inhibitors (for example, PD-1 inhibitors, PD-L1 inhibitors, or CTLA-4 blockade), dendritic cell therapies, adoptive T cell therapy, and tumor vaccines (such as tumor cell vaccines or antigen vaccines). Additional cancer immunotherapies that can be used in the disclosed methods include cytokine therapy (e.g., IL-2), and immune agonists to costimulatory receptors such as 4-1BB or OX40 (e.g., stimulatory anti-4-1BB and anti-OX40 antibodies).

In some embodiments, the subject is administered one or more monoclonal antibodies in addition to the selected HLA nucleic acid(s). Exemplary monoclonal antibodies of use with the disclosed methods include but are not limited to trastuzumab (Herceptin®), pertuzumab (Perjeta®, Omnitarg®), rituximab (Rituxan®), ofatumumab (Arzerra®), ibritumomab (Zevalin®), tositumumab (Bexxar®), alemtuzumab (Campath®), brentuximab (Adcetris®), gemtuzumab (Mylotarg®), labetuzumab (CEA-CIDE®), pemtumomab (Theragyn®), oregovomab (Ovarex®), votumumab (Humaspect®), ramucirumab (Cyramza®), elotuzumab (Empliciti®), daratumumab (Darzalex®), blintumomab (Blincyto®), bevacizumab (Avastin®), panitumumab (Vectibix®), or cetuximab (Erbitux®). The monoclonal antibody is selected based on the type of tumor in the subject, and additional antibodies can be selected by a clinician. In some examples, the antibody is conjugated to a radioisotope or an additional therapeutic agent.

In other embodiments, the subject is administered one or more immune checkpoint inhibitors in addition to the selected HLA nucleic acid(s). Immune checkpoint inhibitors are compounds that inhibit “checkpoint” proteins on T cells, for example, the PD-L1/PD-1 and B7-1/B7-2/CTLA-4 pathways. Checkpoint inhibitors include anti-PD-1 antibodies, such as nivolumab and pembrolizumab and anti-PD-L1 antibodies, such as atezolizumab, avelumab, and durvalumab. Checkpoint inhibitors also include anti-CTLA-4 antibodies, including ipilimumab. In some non-limiting examples, checkpoint inhibitors are utilized to treat melanoma, non-small cell lung carcinoma, renal cell carcinoma, or squamous cell carcinoma of the head and neck.

In additional embodiments, the subject is administered an adoptive T cell therapy, in addition to the selected HLA nucleic acid(s). Adoptive T cell therapy involves using T cells from the subject to recognize cancer cells. In some examples, the T cells are engineered ex vivo to recognize cancer cells (e.g., by introducing a T cell receptor (TCR) or chimeric antigen receptor (CAR) that recognizes a tumor associated antigen) before infusing the T cells back to the subject. In one example, T cells are genetically engineered to express T-cell receptors (TCRs) that recognize a tumor associated antigen or epitope (such as a mutated KRAS-G12D epitope). In other examples, cancer-targeting T cells (such as tumor infiltrating lymphocytes (TIL)) or T cells derived from peripheral blood from the subject are selected ex vivo and then infused back to the subject. In adoptive T cell therapies, the subject typically undergoes a conditioning regimen of lymphodepletion, followed by administration of the modified T cells or selected T cells or TILs.

In one non-limiting example of an adoptive T cell therapy, cultures of TIL are generated from tumor fragments from the subject. Samples of the tumor are also sequenced (for example, using whole exosome sequencing and/or transcriptome sequencing) to identify mutations expressed by the tumor. The TIL cultures are evaluated for reactivity against the identified mutations (tumor neoepitopes). Culture(s) with highest reactivity are selected and expanded. Following a nonmyeloablative lymphodepletion regimen, the TILs are administered to the subject (for example, about 1×10⁷ to 1×10¹² TILs, such as about 1×10⁷, 5×10⁷, 1×10⁸, 5×10⁸, 1×10⁹, 5×10⁹, 1×10¹⁰, 5×10¹⁰, 1×10¹¹, 2×10¹¹, 5×10¹¹, 1×10¹² TILs). In one non-limiting example, the TILs are reactive to a KRAS neoepitope, such as KRAS G12D or KRAS G12V.

In another non-limiting example of an adoptive T cell therapy, samples of the tumor are sequenced (for example, using whole exosome sequencing and/or transcriptome sequencing) to identify mutations expressed by the tumor. T cells (such as autologous T cells) are then genetically modified to express a T cell receptor that recognizes a tumor associated antigen or epitope (for example KRAS-G12D).

Following a nonmyeloablative lymphodepletion regimen, the modified T cells are administered to the subject (for example, about 1×10⁷ to 1×10¹² cells, such as about 1×10⁷, 5×10⁷, 1×10⁸, 5×10⁸, 1×10⁹, 5×10⁹, 1×10¹⁰, 5×10¹⁰, 1×10¹¹, 2×10¹¹, 5×10¹¹, 1×10¹² T cells). In one non-limiting example, the T cells express a T cell receptor that recognizes a KRAS neoepitope, such as KRAS G12D or KRAS G12V.

In further embodiments, the subject is administered a tumor vaccine in addition to the selected HLA nucleic acid(s). Tumor vaccines involve administering one or more tumor antigens to a subject with cancer. In some examples, a tumor-specific neoepitope (such as an epitope with a tumor-specific mutation) is administered to the subject to stimulate an immune response to the tumor.

In some embodiments, a further cancer therapy is administered to the subject in addition to the immunotherapy, including one or more of chemotherapeutic agents, radiation therapy, and surgery. Chemotherapeutic agents are selected based on the type of tumor being treated, and include alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine); antimetabolites such as folic acid analogs (such as methotrexate), pyrimidine analogs (such as 5-FU or cytarabine), and purine analogs, such as mercaptopurine or thioguanine; or natural products, for example vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Additional agents include platinum coordination complexes (such as cis-diamine-dichloroplatinum II, also known as cisplatin), substituted ureas (such as hydroxyurea), methyl hydrazine derivatives (such as procarbazine), and adrenocrotical suppressants (such as mitotane and aminoglutethimide); hormones and antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone). Examples of the most commonly used chemotherapy drugs include adriamycin, melphalan (Alkeran®) Ara-C (cytarabine), carmustine, busulfan, lomustine, carboplatinum, cisplatinum, cyclophosphamide (Cytoxan®), daunorubicin, dacarbazine, 5-fluorouracil, fludarabine, hydroxyurea, idarubicin, ifosfamide, methotrexate, mithramycin, mitomycin, mitoxantrone, nitrogen mustard, paclitaxel (or other taxanes, such as docetaxel), vinblastine, vincristine, VP-16, while newer drugs include gemcitabine (Gemzar®), irinotecan (CPT-11), leustatin, navelbine, imatinib (STI-571), Topotecan (Hycamtin®), capecitabine, and calcitriol.

### III. Methods of Treating Cancer

In some embodiments, the disclosed methods are used without combination with immunotherapy. In some cases, restoring expression and/or function of HLA in a tumor can stimulate the immune system to reject the tumor without administering an immunotherapy (though other cancer therapies, such as chemotherapeutic agents, radiation therapy, and/or surgery, may still be used).

Thus, in some embodiments, the methods include identifying changes in expression of HLA or B2M molecules in a sample of a tumor from a subject. Following selection of an HLA or B2M molecule with altered expression (such as decreased expression or loss of expression) in the tumor, nucleic acid encoding the HLA, and optionally β2-microglobulin (B2M)), is introduced to the tumor cells in vivo. The nucleic acid(s) are introduced to the tumor cells by administering the nucleic acid(s) to the subject as described above.

Methods of identifying changes in HLA expression and/or function in a sample from the tumor, selecting an HLA or B2M molecule with altered expression and/or function, and administering the selected HLA (and/or B2M) nucleic acids to the subject are as described in Sections IIA and IIB.

### EXAMPLES

The present disclosure is illustrated by the following non-limiting Examples.

### Example 1

### Delivery of HLA in Combination with Tumor-Specific T Cells Leads to Tumor Cell Recognition

Direct evidence that HLA defects can lead to the evasion of an effective cancer immunotherapy in humans was demonstrated in a recent case report (Tran et al., NEJM 375:2255-2262, 2016, incorporated herein by reference in its entirety) where a patient with metastatic colorectal cancer was treated with adoptive T-cell therapy of 148 billion T cells containing a high frequency of CD8⁺ T cells that specifically targeted a mutated KRAS (G12D) peptide presented in the context of the HLA-C*08:02 molecule. After treatment, all seven metastatic lesions shrank, and six were eradicated; however one lesion progressed nine months later. This lesion was resected and genomic analysis demonstrated that the lesion contained tumor cells that had LOH at chromosome 6 which encoded the HLA-C*08:02 allele. The loss of HLA-C*08:02 rendered these tumor cells “invisible” to the KRAS-G12D-reactive T cells, since the T cells required this HLA to recognize the mutated KRAS peptide.

### Materials and Methods

HLA Constructs and In Vitro Transcribed (IVT) RNA:

The HLA-C*08:02 allele was synthesized and cloned into the pcDNA3.1+plasmid. The HLA-C*08:02+β2-microglobulin (B2M) construct includes the HLA-C*08:02 gene and the B2M gene cloned into the same vector and separated by a furin-SGSG-P2A sequence that allows for cleavage of the protein into HLA-C*08:02 and B2M after translation. 5′ capped and 3′ polyA tailed RNA was produced using a T7-based IVT kit as per manufacturers' instructions (Ambion and NEB).

Cells:

The MDA-Panc48, HPAC, and Panc-1 pancreatic cells lines all endogenously express the KRAS-G12D mutation, but do not express the HLA-C*08:02 allele. The T cells used in these experiments were allogeneic peripheral blood T cells that have been genetically engineered to express T-cell receptors (TCRs) that recognize a mutated KRAS-G12D epitope presented in the context of HLA-C*08:02. One TCR recognizes the 9 amino acid long (9mer) KRAS G12D peptide GADGVGKSA (SEQ ID NO: 1), while the other recognizes a 10 amino acid long (10mer) KRAS G12D peptide GADGVGKSAL (SEQ ID NO: 2).

RNA Transfection and Coculture Experiment:

Pancreatic cancer cell lines were harvested and then counted and seeded into 24 well plates at 5×10⁴ to 1×10⁵ cells per well. HLA RNA encoding HLA-C*08:02 (SEQ ID NO: 3) and/or B2M (SEQ ID NO: 4) were transfected into the pancreatic cancer cell lines using the lipid based Lipofectamine™ MessengerMAX™ transfection reagent (Invitrogen) at 0.6 μg of RNA per well. T cells expressing either the 9mer or 10mer KRAS-G12D reactive TCRs were then cocultured with the various pancreatic cancer cell lines that had been transfected with nothing (mock transfected) or the indicated HLA construct. After an overnight coculture, the cells were harvested and evaluated for T-cell activation using flow cytometric analysis of the T-cell activation marker 4-1BB.

### Results

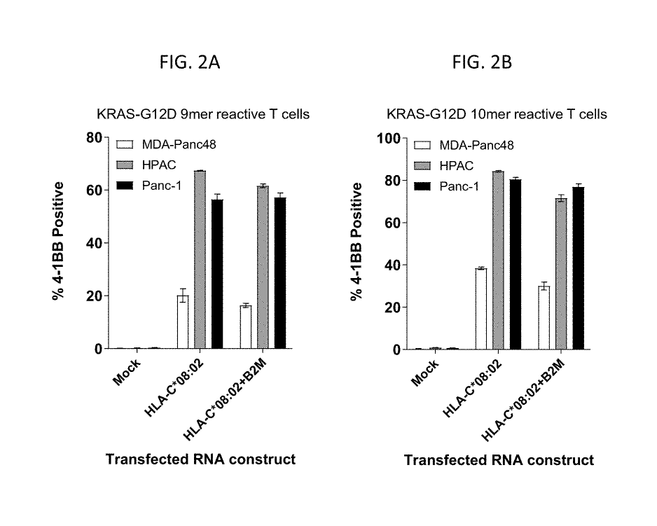

A population of T cells targeting KRAS G12D mutant peptides presented by tumor cells in the context of HLA-C*08:02 was generated. When co-cultured with several different allogeneic pancreatic cancer cell lines naturally expressing the KRAS G12D mutation but not HLA-C*08:02 (mimicking genetic loss of HLA-C*08:02), the KRAS G12D-reactive T cells did not recognize the tumor cells. However the KRAS G12D-reactive T cells recognized these tumor cells when mRNA that encoded HLA-C*08:02 with or without B2M was delivered to the tumor cells using a liposome-based reagent (FIGS. 2A and 2B). These data demonstrate that delivery of the appropriate HLA molecule in combination with a T cell that recognizes a tumor-specific antigen presented by the HLA molecule can lead to tumor cell recognition by the T cells.

### Example 2

### Treatment of a Subject with a Tumor

This example describes an exemplary method for the treatment of a subject with a tumor with a combination of HLA administration and an immunotherapy. However, one skilled in the art will appreciate that methods that deviate from these specific methods can also be used to successfully treat a subject with a tumor.

One or more samples of a tumor (either a primary tumor or tumor metastases) are obtained from a subject. Genomic DNA and/or total RNA are purified from the tumor sample(s). In some examples, genomic DNA and/or total RNA are also purified from one or more non-tumor samples from the subject (such as blood or apheresis sample(s)). Whole exome sequencing, RNA-seq, and/or transcriptome sequencing is carried out on the tumor nucleic acids and non-tumor nucleic acids. HLA allele expression and frequency in the tumor are determined and one or more HLA alleles with decreased expression and/or loss of heterozygosity in the tumor sample are selected.

Tumor infiltrating lymphocytes (TILs) are also isolated from one or more of the tumor samples. Tumor fragments are cultured in complete medium containing high dose (e.g., 6000 IU/ml) IL-2. Neoepitope-reactive T cells are identified by coculturing the TIL cultures with dendritic cells expressing peptides with mutations identified in the subject's tumor by whole exome and transcriptome sequencing. Selected TILs are expanded for administration to the subject. Alternatively, peripheral blood T cells from the subject are modified ex vivo to express T-cell receptors (TCRs) that recognize a tumor associated epitope in the subject.

A nucleic acid encoding the selected HLA allele is prepared. The HLA-encoding nucleic acid (and optionally a nucleic acid encoding B2M) is administered to the subject. The subject also undergoes nonmyeloablative conditioning with cyclophosphamide (e.g., 60 mg/kg for two days) followed by fludarabine (e.g., 25 mg/m² for five days) to lymphodeplete the subject prior to T cell infusion. The subject is then administered the T cells that are reactive to the tumor antigen(s) by infusion with about 1×10⁷ to 1×10¹² total cells, with IL-2 support (e.g., 720,000 IU/kg) and/or other immunotherapy (e.g., immune checkpoint inhibitor) support.

Subjects are monitored (e.g. weekly, biweekly, or monthly) by standard clinical evaluations including physical exams and body weight, and routine clinical labs (hematology and electrolytes). Restaging using PET and CT imaging using RECIST criteria is performed until evidence for tumor progression occurs.

In view of the many possible embodiments to which the principles of the disclosure may be applied, it should be recognized that the illustrated embodiments are only examples, and should not be taken as limiting the scope of the invention. Rather, the scope of the invention is defined by the following claims. We therefore claim as our invention all that comes within the scope and spirit of these claims.

We claim:

1. A method of treating a subject with a tumor, comprising:
(a) obtaining one or more samples comprising tumor cells from the subject;
measuring human leukocyte antigen (HLA) and/or β2-microglobulin (B2M) expression level, genotype, and/or copy number in the one or more samples; and
selecting one or more HLA and/or B2M alleles that have reduced expression, function, and/or copy number;
(b) administering to the subject a nucleic acid encoding the one or more selected HLA and/or B2M alleles; and
(c) administering one or more cancer immunotherapies to the subject.

2. The method of claim 1, further comprising measuring HLA and/or B2M expression level, genotype, and/or copy number in a non-tumor sample from the subject.

3. The method of claim 1, wherein measuring HLA and/or B2M expression level, genotype, and/or copy number comprises one or more of immunoassay, hybridization, or sequencing assays.

4. The method of claim 1, wherein selecting one or more HLA and/or B2M alleles having reduced expression comprises selecting an allele with at least 10% reduced expression compared to a control.

5. The method of claim 1, wherein selecting one or more HLA and/or B2M alleles having reduced copy number comprises selecting an allele with a decreased number of copies compared to a control.

6. The method of claim 1, wherein the nucleic acid encoding the one or more selected HLA and/or B2M alleles is administered to the subject in a viral vector comprising the nucleic acid, an oncolytic virus comprising the nucleic acid, a nanoparticle comprising the nucleic acid, or a liposome comprising the nucleic acid.

7. The method of claim 6, wherein the vector or oncolytic virus comprising the nucleic acid further comprises a promoter operably linked to the nucleic acid.

8. The method of claim 1, wherein the nucleic acid encoding the one or more HLA and/or B2M alleles is locally administered to the tumor in the subject or is administered to the subject systemically.

9. The method of claim 1, wherein the selected HLA nucleic acid encodes an HLA-C*08:02 allele or an HLA-A*11:01 allele.

10. The method of claim 1, wherein the one or more cancer immunotherapies comprises one or more of monoclonal antibodies, immune checkpoint inhibitors, cytokine therapy, agonists to costimulatory molecules, dendritic cell therapies, adoptive T cell therapy, and tumor vaccines.

11. The method of claim 10, wherein the cancer immunotherapy is adoptive T cell therapy.

12. The method of claim 11, wherein the adoptive T cell therapy comprises administering to the subject modified T cells reactive to one or more neoepitopes in the tumor of the subject.

13. The method of claim 12, wherein the neoepitope is KRAS G12D or KRAS G12V.

14. The method of claim 1, wherein the one or more cancer immunotherapies are administered to the subject prior to, simultaneously or substantially simultaneously with, or after administering the nucleic acid encoding the one or more selected HLA and/or B2M alleles.

15. The method of claim 1, wherein the tumor is a primary tumor or a tumor metastasis.

16. The method of claim 1, wherein the tumor is an adrenal tumor, bile duct tumor, bladder tumor, bone tumor, brain tumor, breast tumor, cardiac tumor, cervical tumor, colorectal tumor, endometrial tumor, esophageal tumor, germ cell tumor, gynecologic tumor, head and neck tumor, hepatic tumor, renal tumor, laryngeal tumor, liver tumor, lung tumor, melanoma, neuroblastoma, oral tumor, ovarian tumor, pancreatic tumor, parathyroid tumor, pituitary tumor, prostate tumor, retinoblastoma, rhabdomyosarcoma, non-melanoma skin cancer, gastric tumor, testicular tumor, thyroid tumor, uterine tumor, vaginal tumor, vulval tumor, or Wilms' tumor or wherein the tumor is a hematological malignancy.

17. The method of claim 16, wherein the tumor is a colorectal tumor, a pancreatic tumor, or a non-small cell lung tumor.

18. The method of claim 16, wherein the hematological malignancy is an acute leukemia, a chronic leukemia, T-cell large granular lymphocyte leukemia, polycythemia vera, lymphoma, diffuse large B-cell lymphoma, Hodgkin's lymphoma, non-Hodgkin's lymphoma, mantle cell lymphoma, follicular cell lymphoma, multiple myeloma, Waldenstrom's macroglobulinemia, heavy chain disease, myelodysplastic syndrome, hairy cell leukemia, or myelodysplasia.

19. The method of claim 18, wherein the acute leukemia is acute myeloid leukemia (AML) or acute lymphoblastic leukemia (ALL) or the chronic leukemia is chronic lymphocytic leukemia (CLL) or chronic myeloid leukemia (CML).

20. The method of claim 1, further comprising administering one or more additional therapies, comprising one or more of chemotherapeutic agents, radiation therapy, and surgery.
